(19) United States
(12) Patent Application Publication
(10) Pub. No.: US 2010/0075400 A1
(43) Pub. Date: Mar. 25, 2010

(54) LOW-COST MICROBIAL HABITAT FOR WATER QUALITY ENHANCEMENT AND WAVE MITIGATION

(75) Inventors: Bruce G. Kania, Shepherd, MT (US); Frank M. Stewart, Bozeman, MT (US)

Correspondence Address: ANTOINETTE M. TEASE, P. O. BOX 51016, BILLINGS, MT, 59105, US

(73) Assignee: Fountainhead, LLC, Shephead, MT (US)

(21) Appl. No.: 12/523,733

(22) PCT Filed: Jan. 17, 2008
(86) PCT No.: PCT/US08/51373

§ 371 Date: Jul. 19, 2009

Related U.S. Application Data
(60) Provisional application No. 60/887,802, filed on Feb. 1, 2007.

(51) Int. Cl. C12N 1/00; B63B 35/44; E02B 15/00; C02F 3/00; A01K 61/00; B63B 21/24; E02D 29/00; E99Z 99/00; B32B 3/26

(52) U.S. Cl. 435/243; 114/264; 119/200; 114/294; 405/195.1; 428/317.7

(57) ABSTRACT

A low-cost aquatic structure optimized for water quality enhancement and wave mitigation. The structure is preferably comprised predominantly of recycled polymer scrap or other low-cost, high-surface-area materials, such as jute. The structure may be either buoyant or non-buoyant. Buoyant embodiments of the structure are preferably tip resistant. All embodiments are preferably wave damping. The structure is preferably porous and permeable, and has a large internal surface area for colonization by nutrient-removing microbes. The structure may alternately be used to add nutrients to a water body to promote fish growth for aquaculture applications.

### CROSS-REFERENCE TO RELATED APPLICATION

This application claims priority back to U.S. Patent Application No. 60/887,802, filed on 1 Feb. 2007.

### BACKGROUND OF THE INVENTION

This invention relates to low-cost, man-made structures for use in water. In particular, the invention relates to concentrated surface area, tip-resistant and wave damping floating islands and negatively buoyant structures.

Background art floating platforms are deployed for a wide variety of applications. Floating docks are used by human swimmers for resting and diving. Floating wildlife rafts are used to provide nesting and resting habitat for birds, mammals, reptiles and amphibians. Floating water treatment platforms are used to grow plants and microbes that uptake and convert water-borne contaminants such as excess nutrients and dissolved metals.

All of the structures described above have at least three major deficiencies that are overcome by the present invention. First, background art floating platforms are inherently unstable against tipping when a load is placed near their perimeter (for example, a human swimmer climbing onto a floating dock tends to tilt and submerge the edge of the platform where he is attempting to board). Second, existing-art floating platforms tend to bob and rock excessively when waves are present. Existing designs typically must be “oversized” to counter these motions, which increases the costs of manufacture and deployment. Third, existing designs do not integrate high levels of inexpensive scrap polymers to provide high levels of surface area for colonization by beneficial microbes, which in turn convert pollution-causing nutrients to biomass and nitrogen gas.

The background art is characterized by U.S. Pat. Nos. 5,201,136; 5,224,292; 5,528,856; 5,588,396 5,766,474; 5,980,738; 6,086,755; 6,089,191 and 6,555,219 and U.S. Patent Application Nos. 2003/0051398; 2003/0208954; 2005/0183331; the disclosures of which patents and patent applications are incorporated by reference as if fully set forth herein.

### BRIEF SUMMARY OF THE INVENTION

The purpose of the invention is to provide a high-capacity microbial habitat along with tip-resistance and wave damping for floating islands and submerged structures. Background art floating platforms rely on having a large buoyant mass to resist tipping from edge loads. In preferred embodiment, the present invention uses the weight of trapped water and/or strategically positioned negatively buoyant materials and water-produced drag to counter tipping forces. Therefore, preferred embodiments of the present invention provide enhanced stability with significantly less material mass (and therefore less material cost) than background art designs.

Wave forces have maximum energy at the surface of water bodies, and energy levels decrease with depth. Background art designs for floating platforms typically use deeply submerged floats and/or large mass to provide stability against wave motion. Preferred embodiments of the present invention utilize trapped water weight and water-produced drag to counter wave-induced motion. Therefore, preferred embodiments of the present invention can be made smaller and less costly than existing designs with comparable stability against wave-induced motion. Moreover, by having less dry weight than background art designs, preferred embodiments of the present invention are easier to construct, store, transport and deploy than background art designs.

The islands may also be used as platforms to support water aerators or water circulators. Aerators may be incorporated into the invention for increasing the dissolved oxygen concentration in the water body, which is beneficial for maintaining high growth rates of fish and aquatic insects. Aeration may also be used to increase the dissolved oxygen concentration within the submerged portions of the island body, which may be beneficial for maintaining high nutrient removal rates by microbes that colonize the interior of the island body.

Water circulators may be incorporated into the invention for improving water quality throughout the year. For example, during wintertime in cold climates, water may be circulated from the bottom of the water body to the surface. The relatively warm bottom water is useful for keeping the surface of the water body free of ice, which promotes natural transfer of oxygen and sunlight into the water body. Oxygen and sunlight are required to sustain fish and submerged plants. During summertime in warm climates, water circulation is desirable to slow the growth of free floating algae, by removing the algae from the surface layer, and circulating them to deeper regions that are cooler and have less sunlight.

In preferred embodiments, the present invention is produced in free-form shapes that are more natural in appearance than background art designs. These natural forms are advantageous at locations where aesthetic considerations are important, for example, in wildlife parks.

In preferred embodiments, in order to provide a large surface area for microbial biofilms, the island matrix is designed to have a relatively high ratio of internal surface area to bulk volume. For example, consider a cube of nonwoven polymer matrix having external dimensions of 1 foot on each side, giving a corresponding bulk volume of one cubic foot. Assume that the total surface area of the individual polymer strands within the cube is known to be about 294 square feet. Therefore the ratio of internal surface area to bulk volume is (294 ft²/1 ft³), or 294 square foot of surface area per cubic foot of bulk volume. For the purposes of this disclosure, the term “biomediation quotient” or “BMQ” is defined as the ratio of surface area to bulk volume, in which the bulk volume has dimensions of 1 foot by 1 foot by 1 inch, or 1/12 cubic foot.

In preferred embodiments, the present invention utilizes water-porous and water-permeable materials as a major component of the body of the platform. These materials are preferably assembled in the specific optimized shapes described herein. The combination of these materials and shapes of the floating island components act to minimize tipping and bobbing when the structures are subjected to temporary edge loads or to wave action.

In background art embodiments of floating islands, injected or inserted polymer foam has been utilized to provide adequate buoyancy for the floating structures. This foam is substantially non-permeable to water and gases, and takes up a portion of the internal space of the structure that would otherwise comprise a permeable volume having significant surface area for colonization by beneficial microbes. By using pieces of buoyant polymer scrap as a major component of the present invention, the requirement for including polymer foam for buoyancy is reduced or eliminated. In addition to decreasing material and fabrication costs, the reduction or elimination in buoyant foam from the structure increases the internal volume that is available for colonization by nutrient-removing microbes, thereby increasing the water-quality enhancing properties of the structure.

In a preferred embodiment, the invention is a structure (e.g., a buoyant or non-buoyant island) comprising: a body that has a center and a perimeter and that comprises a positively buoyant, water-porous and water-permeable matrix material that comprises polymer fibers or polymer shreds that are intertwined to form a randomly oriented blanket having an interior and an exterior, at least a portion of said polymer fibers or polymer shreds (e.g., that portion that, in use, is exposed to ultraviolet radiation) preferably being coated with a water-based latex binder or polyurea, said body having a thickened section at said perimeter. Preferably, said body also has a thickened section adjacent to said center. Preferably said randomly oriented blanket has surface areas that are capable of supporting colonization within said interior and along said exterior by microbes, including beneficial microbes that take up and/or convert water-borne contaminants such as excess nutrients and/or dissolved metals.

In another preferred embodiment, the invention is a buoyant island for use in a water body having a water surface, said buoyant island comprising: a platform that has a shape, a center and a perimeter and that comprises a positively buoyant, water-porous and water-permeable matrix material, said platform having a thickened section at said perimeter; wherein, in use, said platform contains a first portion of water that flows through it and a second portion of water that is trapped within said thickened section when said thickened section is lifted above said water surface. Preferably, said platform has a thickened section adjacent to said center. Preferably, said platform has surface areas that are capable of supporting colonization by beneficial microbes. Preferably, said platform has a metacenter and said shape minimizes the shift of said metacenter when tipping loads are imposed on said platform. For the purposes of this disclosure, the term “metacenter” is the point of intersection of a first vertical line that passes through the center of buoyancy of a floating body with a second vertical line that passes through the new center of buoyancy when the body is displaced.

In another preferred embodiment, the invention is a buoyant island comprising: a body that has a perimeter and that comprises a positively buoyant, water-porous and water-permeable matrix material that comprises polyester fibers that are intertwined to form a randomly oriented blanket, said polyester fibers being coated with a water-based latex binder, polyurea or polyurethane, said body having a thinned section at said perimeter and an overhanging lower lip section.

In a further preferred embodiment, the invention is a buoyant island comprising: a first portion that has a perimeter and that comprises a positively buoyant, water-porous and water-permeable matrix material that comprises polymer fibers or polymer shreds that are intertwined to form a randomly oriented blanket, said polymer fibers or polymer shreds being coated with a water-based latex binder, polyurea or polyurethane, said first portion having a thickened section at said perimeter and a center section; and a second portion that is attached to said center section of said first portion, said second portion being negatively buoyant. Preferably, said second portion comprises concrete or stone. Preferably, said positively buoyant, water-porous and water-permeable matrix material has a surface that is capable of supporting colonization by beneficial microbes.

In another preferred embodiment, the invention is an assembly comprising: a plurality of the buoyant structures or buoyant islands disclosed herein; and a plurality of attachment devices that connect each of said buoyant structures or buoyant islands to another of said buoyant structures or buoyant islands in at least two locations. Preferably, each of said attachment devices comprises a rope, a cable or a metal strip, a chain or a cord. Preferably, said water-porous and water-permeable matrix material has a surface that supports colonization by beneficial microbes.

In another preferred embodiment, the invention is a buoyant structure comprising: a body that comprises a positively buoyant, water-porous and water-permeable matrix material that comprises polymer fibers or polymer shreds that are intertwined to form a randomly oriented blanket, said body having an overhanging upper lip section, an undercut center section and an overhanging lower lip section.

In another preferred embodiment, the invention is a structure for installation in a body of water having a water surface, said structure comprising: a plurality of pieces of scrap nonwoven matrix bonded together with a bonding agent or encased in a nonwoven matrix blanket to produce a combination having elements that are operative to provide surfaces for microbial colonization. In a further preferred embodiment, the invention is a structure for installation in a body of water having a water surface, said structure comprising: a plurality of pieces of nonwoven matrix that is comprised of polyester or jute that are encased in one or more blankets of nonwoven matrix that are comprised of polyester or jute. In yet another preferred embodiment, the invention is a plurality of randomly shaped pieces of reground polymer bonded together or encased in a nonwoven matrix blanket that is operative to provide a surface for microbial colonization. Preferably, said combination is positively buoyant. Preferably, said combination is negatively buoyant.

Preferably, the structure has an interior having an interior surface area and an outer surface having an outer surface area, a portion of which outer surface area is above the water surface, and said interior surface area is a multiple of said outer surface area. Preferably, the structure has an interior having an interior surface area and an outer surface having an outer surface area, and said interior surface area is greater than said outer surface area. Preferably, the structure is further comprised of polymer scrap pieces and said polymer scrap pieces are comprised of a combination of polymer fibers and a polymer foam. Preferably, the structure is further comprised of two layers of nonwoven polymer matrix, said polymer scrap pieces are arranged in a layer, and said layer of polymer scrap pieces is sandwiched between said two layers of nonwoven polymer matrix. Preferably, the structure is further comprised of multiple alternating polymer scrap piece layers and nonwoven polymer matrix layers. Preferably, said polymer scrap pieces are comprised of unsorted materials. Preferably, said polymer scrap pieces are comprised of materials having a specific gravity less than 1.0. Preferably, said polymer scrap pieces form a combined mixture and polymer scrap pieces are comprised of materials having a range of specific gravities, such that said combined mixture of polymer scrap pieces has a net positive buoyancy. Preferably, said polymer scrap pieces are comprised of materials having a specific gravity greater than 1.0. Preferably, said polymer scrap pieces form a combined mixture and said polymer pieces are comprised of materials having a range of specific gravities, such that said combined mixture of polymer scrap pieces has a net negative buoyancy.

In yet another embodiment, the invention is a structure for installation in an aqueous environment comprising: a porous containment bag; and a plurality of pieces of scrap polymer that are encased within said porous containment bag. Preferably, at least some of said pieces of scrap polymer have a specific gravity that is less than that of water, and the structure has a positive buoyancy. Preferably, at least some of said pieces of scrap polymer have a specific gravity that is greater than that of water, and the structure has a negative buoyancy.

In another preferred embodiment, the invention is a negatively buoyant structure comprising: a plurality of polymer pieces having a total surface area and a bulk volume and having a total surface area to bulk volume ratio of at least 200 that, together, are operative to provide biomimetic replication of a natural coral formation in saltwater or a stone formation in freshwater, having cavities and crevices for use by aquatic animal life for hiding, resting or feeding. In another preferred embodiment, the invention is a permeable and negatively buoyant structure for installation in a water body having a bottom, said structure comprising: a plurality of polymer pieces having a total surface area and a bulk volume and having a total surface area to a bulk volume ratio of at least 200, that, together, are operative to anchor a floating island or to tether another floating object to the bottom, thereby allowing the anchoring of a floating object when the bottom of the water body is soft or otherwise unsuitable for conventional anchors, the permeability of said structure providing additional drag when said object is pulled through the water body, thereby enhancing the anchoring properties of said structure.

In another preferred embodiment, the invention is a buoyant structure comprising: a body that is selected from the group consisting of: (1) a first portion having a periphery and comprising a positively buoyant, water-porous and water-permeable matrix material, and a second portion comprising a pontoon member that is disposed at said periphery of said first portion, (2) a first portion comprising a platform having a periphery and a center section that is comprised of a positively buoyant, water-porous and water-permeable matrix material, a second portion comprising a pontoon member that is disposed at said periphery of said first portion, and a third portion that is attached to said center section, (3) a first portion having a periphery and comprising a platform having a center section that is comprised of a positively buoyant, water-porous and water-permeable matrix material, a second portion that is disposed at said periphery of said first portion, said second portion being thinner in cross section than said center section, and a third portion that is attached to said center section, (4) a first portion having a periphery and comprising a platform having a center section that is comprised of a positively buoyant, water-porous and water-permeable matrix material, a second portion comprising a pontoon member that is disposed at said periphery of said first portion, and a third portion that is attached to said center section, said third portion being negatively buoyant, (5) a first discrete portion comprising a positively buoyant, water-porous and water-permeable matrix material, and a second discrete portion comprising said positively buoyant, water-porous and water-permeable matrix material, said discrete portions not being in contact with one another, and (6) a middle portion that is comprised of a positively buoyant, water-porous and water-permeable matrix material, said middle portion having a periphery, a top portion that is comprised of said positively buoyant, water-porous and water-permeable matrix material, said top portion extending radially beyond said periphery, and a bottom portion that is comprised of said positively buoyant, water-porous and water-permeable matrix material, said bottom portion extending radially beyond said periphery; and a plurality of attachment means that connect said portions to one another in at least two places; wherein, in use, each of said portions contains a first quantity of water that flows through it and/or a second quantity of water that is trapped within it when it is lifted above said water surface. In this embodiment, the “positively buoyant, water-porous and water-permeable matrix material” may be positively buoyant due to the buoyancy of the nonwoven polymer fibers or polymer shreds, and/or it may be positively buoyant due to buoyant polymer foam that is added to said matrix.

In another preferred embodiment, the invention is a floating island for installation in a water body, said floating island comprising: three first layers, each first layer comprising a nonwoven polymer matrix; and two second layers, each second layer comprising a plurality of scrap polymer pieces; wherein each of said second layers is disposed between two of said first layers. Preferably, the floating island further comprises: an inlet pipe that is extendable into the water body; a water pump that is operative to move water into said inlet pipe; a solar collector that is operative to supply power to said water pump; and a discharge line for distributing said water over the uppermost of said first layers.

In another preferred embodiment, the invention is a simulated coral reef comprising: a plurality of scrap polymer pieces that are bonded together to produce a non-buoyant body; and an injection system for injecting water and/or air into said non-buoyant body. Preferably, said non-buoyant body has cavities. Preferably, the simulated coral reef further comprises a bag of dry cement that is disposed in one of said cavities.

In another preferred embodiment, the invention is a polymer scrap structure comprising: a plurality of scrap polymer pieces that are bonded together with polyurea or polyethylene to form a body having cavities; and a gas-impermeable top coat comprised of polyurea or polyethylene. In another preferred embodiment, the invention is a floating island comprising: a sheet of nonwoven matrix having a top side and a bottom side; a first plurality of scrap polymer pieces that are attached to said top side to produce a growth platform, said growth platform comprising a perimeter lip and having capillary tubes that are filled with a hydrophilic material; a second plurality of scrap polymer pieces that are attached to said bottom side; and a plant growth medium that is disposed on said growth platform, said plant growth medium being in communication with said hydrophilic material in said capillary tubes. Preferably, the floating island further comprises: matrix scrap pieces that are disposed within said second plurality of scrap polymer pieces.

In another preferred embodiment, the invention is a floating island comprising: a single bottom layer that is comprised of nonwoven matrix; a middle portion that is comprised of scrap nonwoven matrix or another polymer material; and a top blanket of sod, sod impregnated jute or sod impregnated polymer blanket. Preferably, said nonwoven matrix is comprised of a natural nonwoven material. Preferably, said natural nonwoven material is selected from the group consisting of coir, jute, hemp and cotton.

In another embodiment, the invention is an island that is manufactured in a “sandwich” configuration, using relatively thin layers of nonwoven matrix that are separated by relatively thick layers of polymer strands, polymer chips or polymer shreds. The nonwoven matrix may be comprised of 1-inch thick nonwoven polyester, polypropylene, or polyethylene fibers. Alternately, sheets of extruded polymer foam may be used in place of nonwoven matrix. The pore spaces within the sheets of extruded foam may be closed-cell, open-cell, or a combination of closed and open cell foam. The polymer pieces may be comprised of recycled scrap materials. Examples of suitable scrap materials include HDPE (high density polyethylene) milk jugs and PETE (polyethylene terephthalate) soft drink bottles. Polymer jugs and bottles are commonly recycled by grinding and passing the resulting pieces through a ½-inch screen, whereby the maximum dimensions of the resulting scrap pieces are approximately ½-inch wide, ½-inch long, and the thickness of the original polymer container wall. The shapes of the scrap pieces may optionally be optimized for such applications by cutting the pieces into custom shapes and sizes, such as relatively long, narrow strips that may be mechanically intertwined and/or bonded with a latex, polyurea or polyurethane coating to form a blanket having a large available internal surface area for colonization by beneficial microbes. One example of more preferred strip dimensions would be 1/16-inch wide, 3 inches long, and having a thickness of the original wall thickness of the recycled polymer container from which the scrap was produced. Optionally, the strips may be intentionally formed using cutting blades that produce jagged edges on the strips. These jagged edges may help the strips to lock together when intertwined into a nonwoven blanket. The jagged edges may also maximize available surface area for microbial colonization on each strip. These jagged-edge strips biomimic the roots and other organic debris that comprise some natural floating islands.

In any of the embodiments described above, the floating island or other structure may be fabricated from nonwoven polymer matrix (or foam sheets) and pieces of polymer strands, chips or shreds, and a coating that is applied only to the outside surface of the floating island or other structure. Said coating may be comprised of polyurea, polyurethane, latex, rubber, or any other similar material that protects the polymer material from ultraviolet (UV) light degradation, while bonding the materials together. In these embodiments, the size of the scrap pieces and the size of the openings within the nonwoven matrix (or foam sheets) are chosen so as to be compatible, in order to produce a structure in which the internal polymer pieces cannot escape from the structure through the openings in the nonwoven matrix (or foam sheets), yet water and gases are able to pass through the structure.

Shredded pieces of automobile tires or other objects comprised from natural or synthetic rubber may be used as polymer shreds in both the floating and non-floating embodiments. Although junk automobile tires have been bundled together in the background art to create artificial reefs in previous inventions, the present invention preferably utilizes shredded pieces rather than whole tires. The shredded pieces provide much greater surface area per unit mass than whole tires, making the pieces more suitable for colonization by beneficial microbes. The structures may optionally be used as support bases for aerators or water circulators, which are used to enhance water quality.

In another embodiment, the entire structure is comprised of shredded polymer pieces that may be manufactured from recycled scrap. In this embodiment, the polymer pieces may be bonded together with a spray coating of polyurea or polyurethane. Alternately, the shredded pieces may be contained within a permeable bag comprised of polymer, nylon, or other suitably porous material. On example of a suitable material is extruded polyethylene mesh having a screen opening size that prevents the escape of the shredded polymer pieces contained within a bag that is made from the mesh material. One such mesh material is available from McMaster-Carr of Los Angeles, Calif. (part number 9314T29). This embodiment may be used as a conventional, buoyant floating island.

Alternately, all embodiments may be manufactured so as to be negatively buoyant. In the negatively-buoyant configuration, the structure resembles a simulated coral reef, which rests on the bottom of the water body. The simulated coral reef may be injected with air and/or water to promote microbial removal of dissolved nutrients, and to supply oxygen to fish and other aquatic animals that reside around and within the structure. In a similar embodiment that is optimized for aquaculture, nutrients, organic carbon, and/or other materials may be added to the injection water and injected into the structure in order to promote the growth of plankton and microbes, thereby stimulating the food chain, and resulting in increased production of fish or other commercial aquatic products.

For the embodiments that comprise polymer scrap, chips or shreds, the cost of the polymer materials may be minimized by utilizing a blend of various polymer scraps that are available at relatively cost from recyclers. These unsorted blends may be comprised of any combination of polymer materials. Unsorted polymer scrap blends are commonly available at lower cost than sorted scrap, because they currently have limited market potential compared to sorted polymers.

When scrap blends are used in preferred positively buoyant embodiments of the present invention, it may be advantageous to utilize partially sorted blends that are comprised solely or primarily of materials having a specific gravity less than 1.0. These scrap blends have positive net buoyancy, and are available at lower cost than scrap that has been sorted to comprise a single polymer material.

The size and shape of the polymer scrap chips may be varied in the recycling process so as to provide an optimum combination of advantageous qualities. Advantageous qualities may include greater surface area for microbial colonization, increased porosity and stiffness for plant root support, water permeability, and the ability to be contained easily within a matrix “sandwich” without escaping.

For a given mass of polymer scrap, the surface area available for microbial colonization generally increases as the size of the individual chip size decreases (i.e., the ratio of surface area to volume becomes greater as the chip size becomes smaller). Also, chips made from thin stock (such as scrap milk jugs) generally have more surface area per unit mass than chips produced from thick stock (such as automobile bumpers). Therefore, in applications of the present invention in which high internal surface area is important, small or thin chips may be preferred over large or thick chips.

The unit surface area of one sample of blended scrap was measured. This material was a low-cost blend of polyethylene and polypropylene that was run through a grinder with a one-half inch screen. Based on measurements of representative chips, the estimated surface area for these chips was 32.2 square feet of surface area per cubic foot of chips, or 1.2 square feet of surface area per pound of chips. This is equivalent to 2.7 square feet for a volume that is 1 square foot by 1 inch thick, or roughly one-tenth the BMQ of the matrix. If the chips were run through a one-quarter-inch screen, the approximate surface area of these chips would be 5.4 square feet for a volume that is one square foot by one inch thick, or roughly one-fifth the BMQ of the matrix.

Further aspects of the invention will become apparent from consideration of the drawings and the ensuing description of preferred embodiments of the invention. A person skilled in the art will realize that other embodiments of the invention are possible and that the details of the invention can be modified in a number of respects, all without departing from the concept. Thus, the following drawings and description are to be regarded as illustrative in nature and not restrictive.

### BRIEF DESCRIPTION OF THE SEVERAL VIEWS OF THE DRAWINGS

The features of the invention will be better understood by reference to the accompanying drawings which illustrate presently preferred embodiments of the invention. In the drawings:

FIG. 1 is a side (elevation) cross-section view of a preferred embodiment of a floating island in accordance with the invention.

FIG. 2 is another side cross-section view of the embodiment of FIG. 1.

FIG. 3 is a side cross-section view of another preferred embodiment of the invention, which has a thickened center section.

FIG. 4 is a schematic side elevation view of another preferred embodiment of the invention.

FIG. 5 is a schematic side cross-section view of another preferred embodiment of the invention.

FIG. 6 is a side cross-section view of yet another preferred embodiment of the invention.

FIG. 7 is a top plan view of another preferred embodiment of the invention.

FIG. 8 is a side cross-section view of yet another preferred embodiment of the invention.

FIG. 9 is a side cross-section view of a sandwich configuration buoyant island in accordance with another preferred embodiment of the invention.

FIG. 10 is a side cross-section view of a simulated coral reef structure in accordance with another preferred embodiment of the invention.

FIG. 11 is a side cross-section view of a polymer scrap floating island in accordance with another preferred embodiment of the invention.

FIG. 12 is a side cross-section view of an island comprising polymer scrap and growth medium in accordance with another preferred embodiment of the invention.

FIG. 13 is a side cross-section view of a three-layer island with overhanging top blanket.

The following reference numerals are used to indicate the parts and environment of the invention on the drawings:

1 floating island, buoyant island, buoyant structure, structure, platform

2 body of water, water body

3 porous and permeable matrix material, nonwoven matrix, matrix

4 portion of island above waterline

5 portion of island below waterline

6 vertical load

7 portion of island depressed by vertical load, first portion

8 downward directional arrow, downward arrow

9 portion of island lifted by vertical load, second portion, uplifted portion

10 upward directional arrow, upward arrow

11 thickened center section, center section

12 arrows depicting up-and-down wave forces, up and clown arrows

13 arrows depicting rocking motion wave forces, rocking arrows

14 thin edge zone

15 negatively buoyant region

16 attachment devices

17 overhanging top lip section

18 undercut center section

19 overhanging lower lip section, overhanging lip feature

20 habitat area, habitat feature

21 fish

22 sandwich configuration island, sandwich island

23 scrap polymer pieces, scrap pieces

24 solar panel

25 water pump

26 inlet pipe

27 discharge lines

28 simulated coral reef structure

29 injection system

30 cavities

31 polymer scrap island, polymer scrap structure

32 impermeable top coat

33 growth medium

34 capillary tubes

35 perimeter lip

36 scrap matrix pieces

40 intake arrows

41 bottom layer

42 middle portion

43 top blanket

44 polyurethane foam

### DETAILED DESCRIPTION OF THE INVENTION

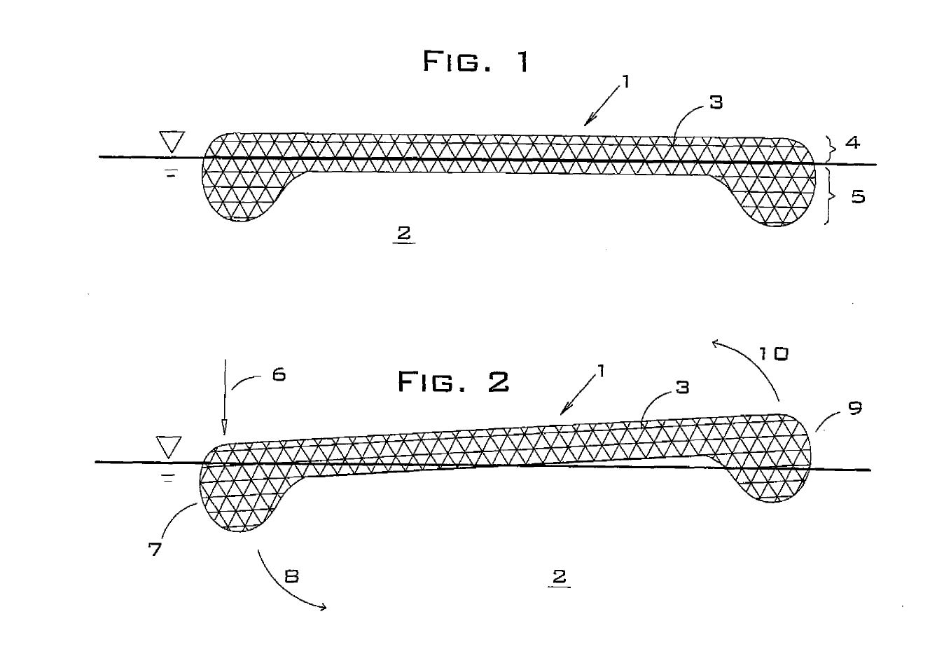

Referring to FIG. 1, a preferred embodiment of floating island 1 is shown floating in a normal position within a body of water 2. In this embodiment, structure 1 is substantially round in shape when viewed in plan from above. This embodiment is referred to herein as the “pontoon design,” from the thickened, pontoon-like shape (in cross section) of the section of the body of floating island 1 at its perimeter. FIG. 2 is a side cross-section view of the same embodiment, shown when the invention is being subjected to a temporary perimeter load.

As shown in FIG. 1, floating island 1 is comprised of a water-porous and water-permeable matrix material 3. Portion 4 of floating island 1 is above water line, and portion 5 is below the waterline. The pore spaces of matrix 3 within the above-waterline portion 4 are filled with air, and the pore spaces of matrix 3 that are within the below-waterline portion 5 are filled with water. In this embodiment, structure 1 floats because the fibers comprising matrix 3 have a density that is less than the density of water. Alternately, supplemental buoyancy may be provided by providing injected polymer foam floatation (not shown). Matrix 3 may be comprised of polymers or natural materials.

In a preferred embodiment, matrix 3 is comprised of polyester fibers that are intertwined to form a randomly oriented web or “blanket,” preferably with a standard thickness and width. While smaller islands may be made of a single piece and thickness of matrix, the dimensions of a larger island body are set by attaching multiple pieces of matrix 3 side-by-side and/or vertically. In one preferred embodiment, matrix 3 is comprised of 200-denier polyester fibers that are intertwined to form a blanket approximately 1¾ inch thick by 56 inches wide.

Preferably, matrix 3 is produced in a continuous strip and is cut into portions having lengths of approximately 90 feet for shipping. The nominal weight of the blanket is preferably 41 ounces per square yard. The nominal weight of the polyester fibers within the blanket is preferably 26 ounces per square yard. A water-based latex binder is preferably baked onto the fibers to increase the stiffness and durability of the blanket. The characteristics of matrix 3 can be adjusted by varying the construction materials and manufacturing process. For example, the diameter of the fibers may be varied from approximately 6 to 300 denier. Coarse fibers result in a relatively stiff matrix with relatively small surface area for colonizing microbes, and fine fibers result in a relatively flexible matrix with a relatively large surface area for colonizing microbes. The latex binder can be applied relatively lightly or relatively heavily to vary the durability and weight of the matrix, and dye or pigment can be added to the binder to produce a specific color of matrix.

The thickness of the blanket can be adjusted from approximately ¼-inch to 2 inches using conventional manufacturing techniques. It is anticipated that thicker blankets will be produced in the future, and these thicker blankets (for example, 3 to 12 inches) will be used as island body material when they become available. The blankets with integral latex binder may be purchased as a manufactured item. One manufacturer of suitable matrix material is Americo Manufacturing Company, Inc. of Acworth, Ga. Alternately, matrix 3 may be comprised of natural nonwoven materials such as coir, jute, hemp or cotton.

Referring to FIG. 2, the position of floating island 1 is illustrated just after a significant vertical load 6 has been applied to an edge of structure 1. Load 6 produces a tipping moment on island 1. The tipping moment causes first portion 7 of island 1 to move in the direction of downward arrow 8, deeper into water body 2. Similarly, the tipping moment causes second portion 9 of island 1 to move in the direction of upward arrow 10, rising above waterline.

In a preferred embodiment, floating island 1 comprises three features that resist the tipping moment produced by vertical load 6. First, the extra weight of matrix 3 due to the thickened perimeter of uplifted portion 9 provides a resisting moment arm force that is greater than would be provided by a structure without a thickened perimeter. Second, water that is trapped within uplifted portion 9 takes some time to drain from permeable matrix 3 due to the surface tension between the water and the fibers of matrix 3. The trapped water adds extra weight to uplifted portion 9 that is raised above waterline, and this extra weight increases the resisting moment arm. Third, the water-porous and water-permeable nature of matrix 3 causes water to flow through matrix 3 whenever floating island 1 is moved through water body 2. The water movement through the matrix fibers produces drag forces that resist the movement of floating island 1 within water body 2. In FIG. 2, first portion 7 of island 1 that is being moved in the direction of downward arrow 8 encounters significant drag as it is submerged in water body 2, thereby resisting rotational movement due to the tipping moment. The buoyancy of first portion 7 that is being submerged also resists rotational movement.

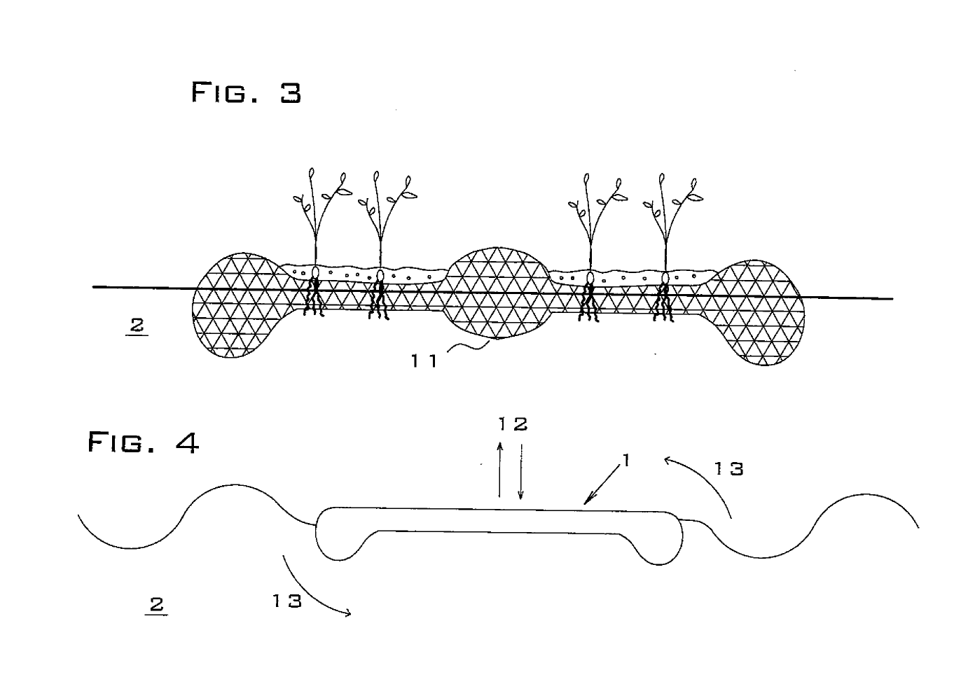

Referring to FIG. 3, another preferred embodiment of the invention having thickened center section 11 is presented. This embodiment has the same three anti-tipping features described for the embodiment of FIGS. 1 and 2. In addition, center section 11 of the embodiment shown in FIG. 3 provides additional moment arm and water drag to resist tipping due to edge loads.

Preferred embodiments of the invention are also resistant to movements due to wave action. Referring to FIG. 4, waves produce both up-and-down forces (shown by arrows 12) and rocking forces (shown by rocking arrows 13) on floating island 1. Both of these forces are resisted by floating island 1. The weight of trapped water in portions of floating island 1 that are lifted above waterline resists such upward motion, while drag forces produced by water flowing through the matrix 3 of moving, submerged portions of the floating islands resist both vertical and rocking motion induced by wave forces. In addition, as wave water is forced into and through the porous and permeable matrix 3 of floating island 1, wave energy is dissipated and reflected, thereby reducing the magnitude of the wave height and energy.

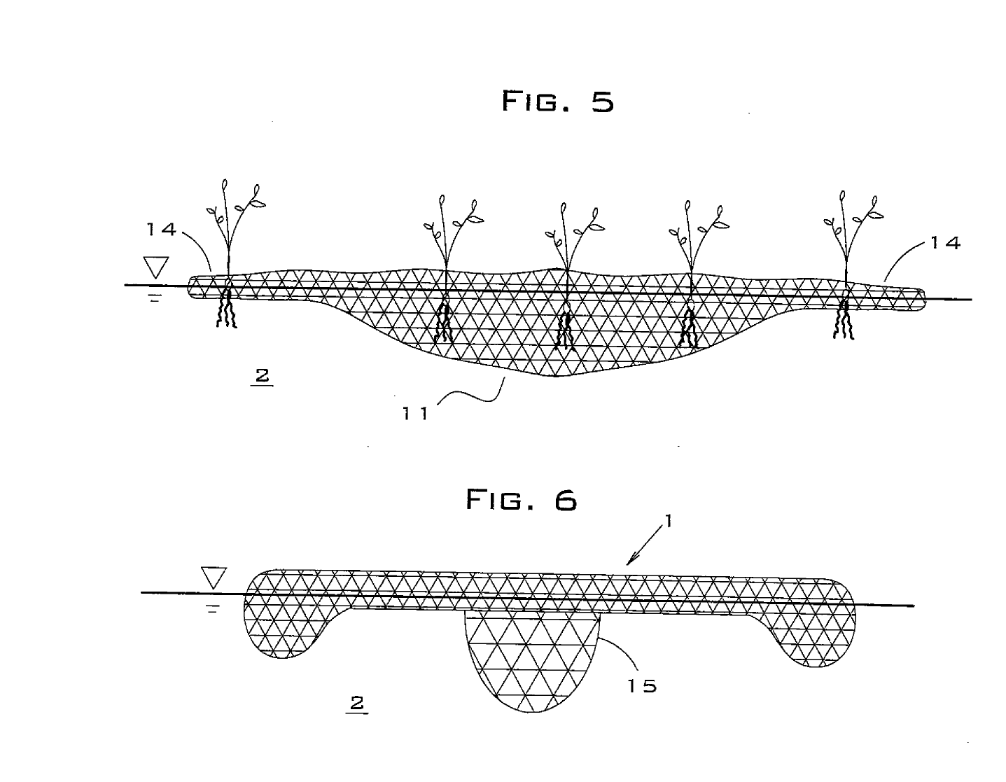

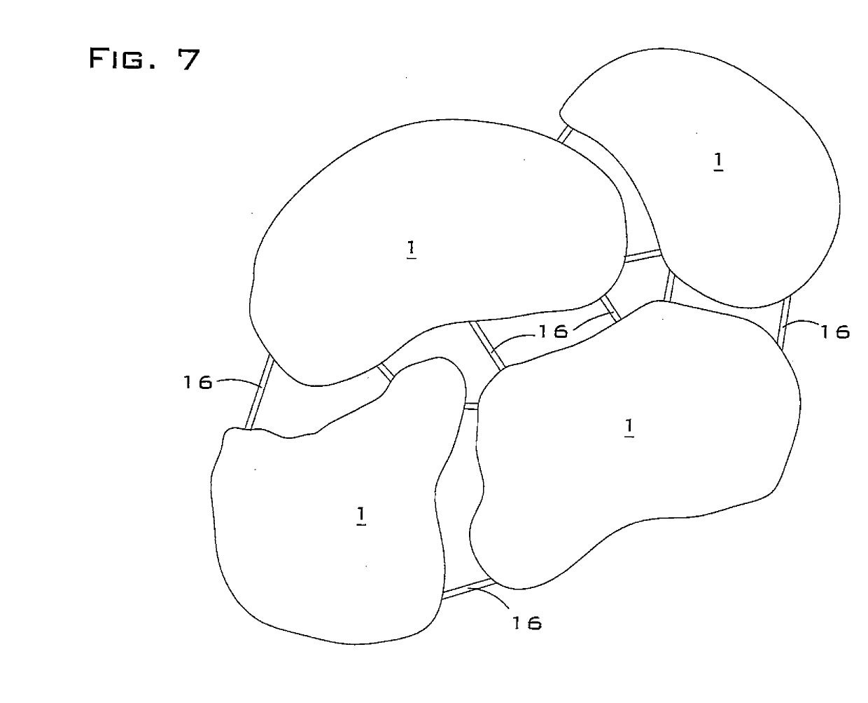

Referring to FIG. 5, another preferred embodiment of the invention is presented. This embodiment mimics the shape of some natural islands that were investigated in Michigan and Wisconsin by the applicants during 2004. In this embodiment, large, water-saturated center section 11 provides a heavy, low center of gravity that resists vertical motion, while thin edge zones 14 provides wave-damping action due to their relatively large surface areas, which serves as a breakwater against incident waves.

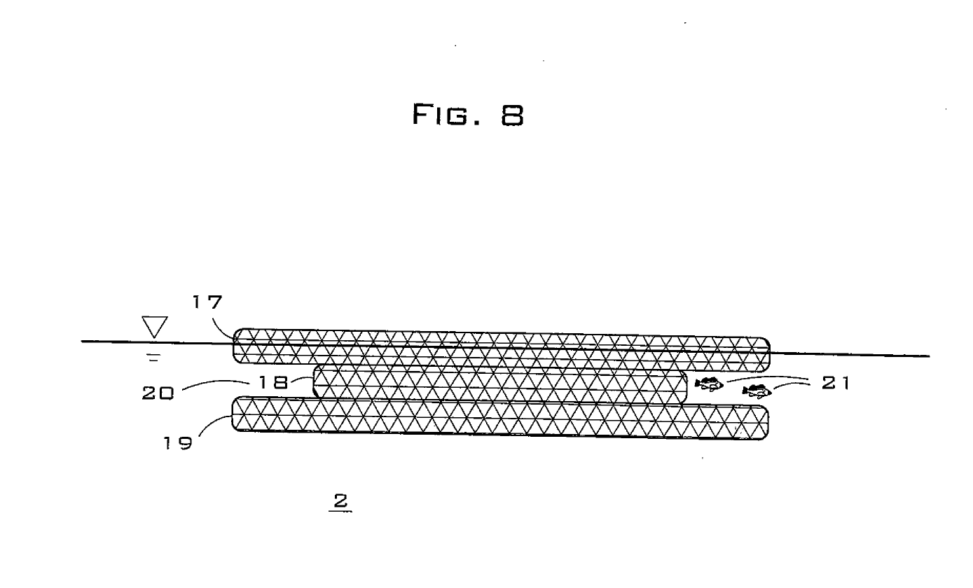

Referring to FIG. 6, yet another preferred embodiment of the invention is presented. This embodiment incorporates a negatively buoyant region 15 within the body of floating island 1. Negatively buoyant region 15 may be comprised of permeable and porous matrix material that is negatively buoyant. Nonwoven polyester is an example of a preferred negatively buoyant matrix material. Alternately, negatively buoyant region 15 may be comprised of negatively buoyant material such as concrete or stone that is placed within the matrix material making up the body of floating island 1. Negatively buoyant region 15 serves as a keel to lower the center of gravity of floating island 1. This keel effect, in combination with the porous and permeable matrix comprising region 15, further enhances island stability.

Referring to FIG. 7, another preferred embodiment of the invention is presented. In this embodiment, outrigger floating islands 1 are used to provide an anti-tipping feature. As shown in the drawing, separate outrigger floating islands 1, ideally of the same porous and permeable matrix construction, are connected to one other with attachment devices 16. This arrangement allows for designable levels of water-produced drag. In a preferred embodiment, such floating islands 1 are joined in at least two locations, preferably towards the opposing ends of the smaller floating island 1. In the event floating islands 1 of similar size are joined in this fashion, preferred attachment points would again tend to correspond with opposing ends of each floating island 1, to allow for utilization of attachment devices 16 to provide a physical barrier to island tipping. Attachment devices 16 may be comprised of any suitably strong and durable material such as rope, cable, or metal strips.

Referring to FIG. 8, yet another preferred embodiment of the invention is presented. In this embodiment, an overhanging lip feature 19, preferably fabricated from the same water-porous and water-permeable matrix material, is incorporated into, preferably, the lowest portion of an island. Besides adding a designable level of tip resisting drag, such a horizontal yo-yo shaped design provides additional underwater habitat feature 20. As shown in FIG. 8, this embodiment comprises overhanging upper lip section 17, undercut center section 18, and overhanging lower lip section 19. Habitat area 20 that is produced by the undercut center section 18 may be utilized by fish 21 and other wildlife species.

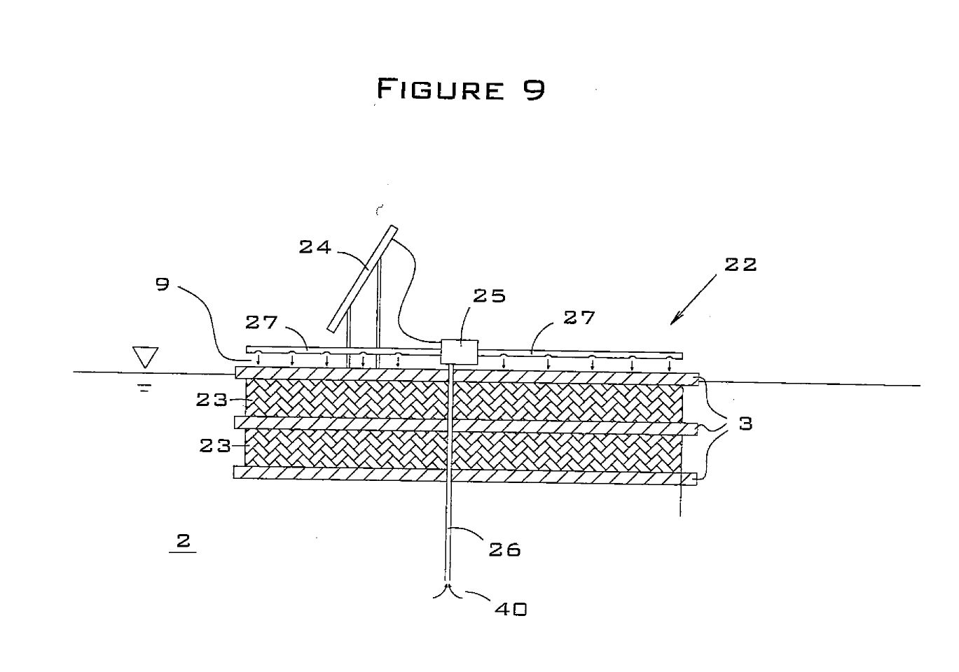

Referring to FIG. 9, a sandwich configuration island 22 is illustrated that is preferably comprised of three layers of nonwoven polymer matrix 3 and two layers of recycled scrap polymer pieces 23 although other numbers of layers may be used. Also shown are optional water circulation components that consist of solar panel 24, water pump 25, inlet pipe 26 and discharge lines 27. In this embodiment, nutrient-bearing water from water body 2 is drawn up (shown by intake arrows 40) through inlet pipe 26 by means of pump 25, and then sprinkled across the surface of sandwich island 22 via discharge lines 27. The water percolates through the layers of porous matrix 3 and scrap pieces 23, where nutrients are removed by microbes colonizing the internal surfaces of matrix 3 and scrap pieces 23. The island may be made in any desired thickness by adjusting the thickness of the layers comprising scrap pieces 23 and the layers comprising matrix 3, and by adjusting the number of alternating layers of scrap pieces 23 and matrix 3.

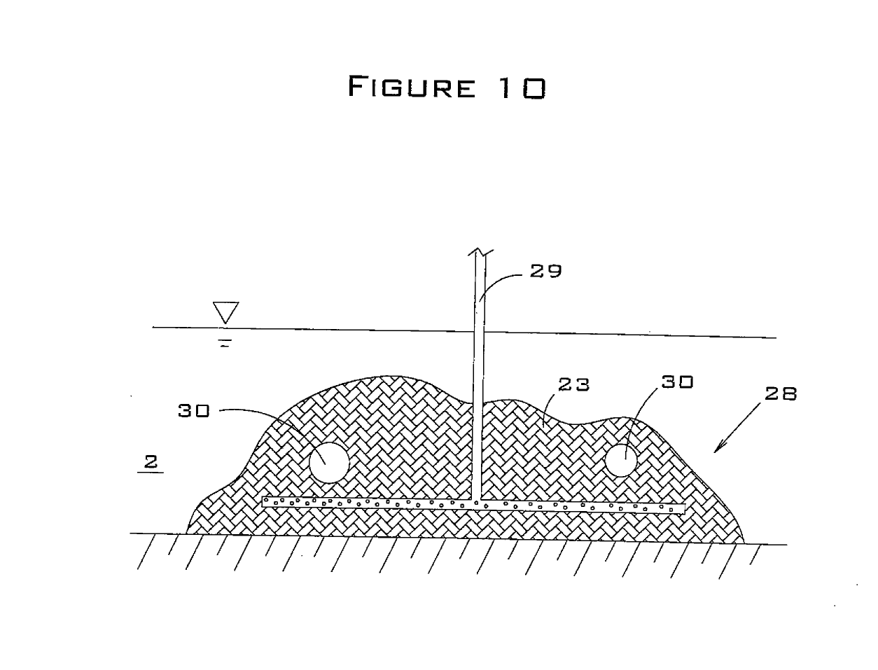

Referring to FIG. 10, simulated coral reef structure 28 is illustrated in accordance with a preferred embodiment of the invention. In this embodiment, simulated coral reef structure 28 is negatively buoyant and rests on the bottom of water body 2. Structure 28 may be used to dissipate wave energy in shallow waters, and may also be used as a water-quality enhancement device. Structure 28 is comprised primarily of scrap polymer pieces 23. Scrap pieces 23 may be bonded together by application of a sprayed-on polyurea or a latex binder (not shown). Scrap pieces 23 may alternately be bonded together by partially melting the pieces 23 with heat. Also shown in FIG. 10 is optional injection system 29. Injection system 29 is used to discharge nutrient-rich water and/or air into the body of the structure 28, thereby promoting growth of colonizing microbes and/or increasing the oxygen supply for fish and other aquatic animals residing within and around structure 28. Injection system 29 is supplied with water and/or air from an external pump (not shown). Optional cavities 30 are also shown. Cavities 30 may be used as resting, feeding, or hiding areas for fish and other animals. Alternately, cavities 30 may be used to insert stones or other heavy objects, thereby increasing the negative buoyancy of structure 28. Structure 28 may optionally comprise bags of dry cement (not shown) that absorb water and cure in place after structure 28 is deployed, thereby adding negative buoyancy. Structure 28 may optionally be used as an anchor for floating islands or other floating objects (not shown).

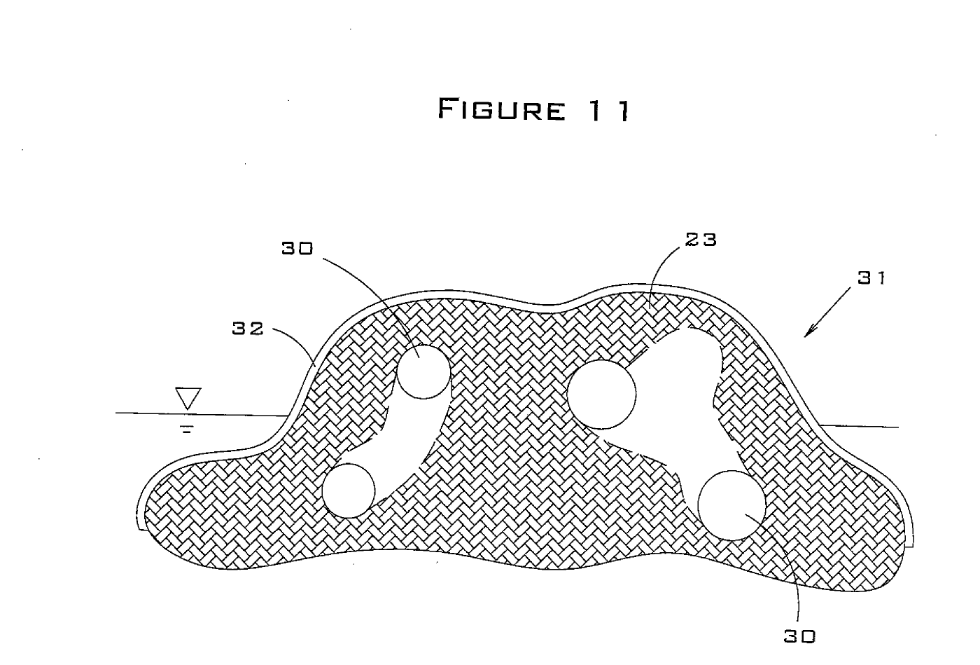

Referring to FIG. 11, a polymer scrap structure 31 is illustrated in accordance with another preferred embodiment of the invention. This embodiment is comprised of scrap polymer pieces 23 that are bonded together with sprayed-on polyurea or polyurethane. Buoyancy may be provided by scrap polymer pieces 23, if the polymer used has a density less than that of water. Additional optional buoyancy may be supplied by polyurethane or thermoplastic foam (not shown). The optional polymer foam may be either injected and cured in place, or it may be provided by preformed foam pieces that are inserted into the body of polymer scrap structure 31 during manufacture. Optionally, scrap pieces of polymer foam may be mixed with scrap pieces of polymer chips to provide the necessary characteristics of permeability, concentrated surface area and buoyancy. Another optional source of buoyancy is gasses that are trapped within the body of structure 31. These gasses may be injected into the island by aeration, or alternately, they may be produced by microbes that colonize the interior of the island body. Optional impermeable top coat 32 may be installed on the outer surface of the island to enhance the gas-trapping abilities of structure 31. Gas-impermeable top coat 32 may be comprised of polyurea or polyurethane. Structure 31 may also have cavities 30 that have openings either above or below waterline (or both), and may be used as habitat for waterfowl, fish, or other aquatic animals.

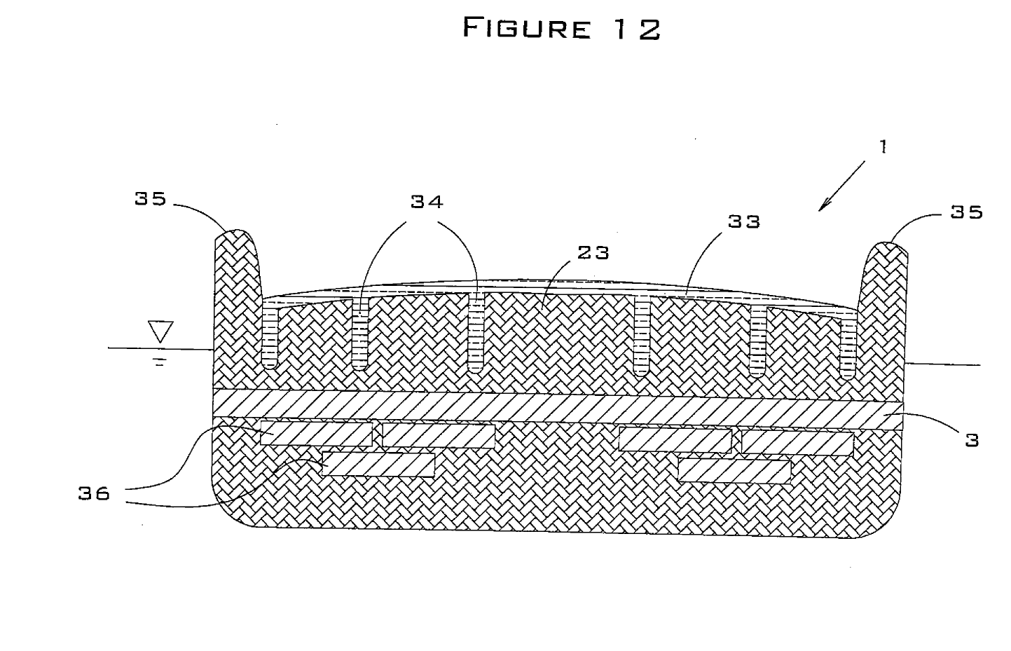

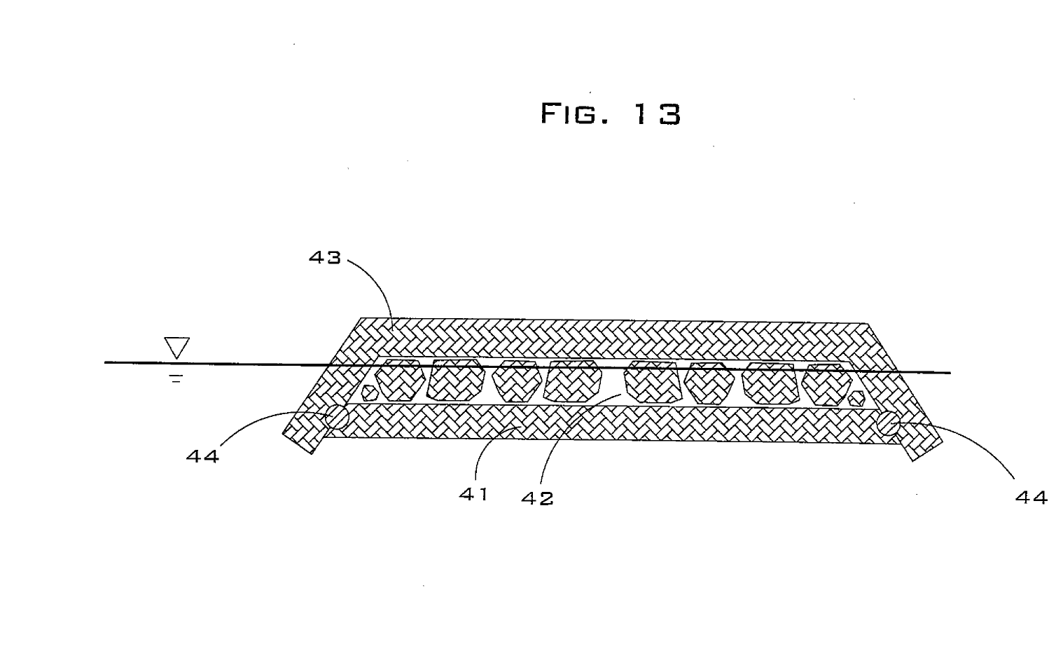

Referring to FIG. 12, another preferred embodiment of floating island 1 is illustrated that comprises a sheet of nonwoven matrix 3, scrap polymer pieces 23 that are bonded to both top and bottom sides of matrix 3, growth medium 33 and capillary tubes 34. Scrap polymer pieces 23 may be bonded together with polyurea or polyurethane. Growth medium 33 may be comprised of BIOMIX™, which is available from Floating Island International, Inc. of Shepherd, Mont., or any other suitable hydrophilic plant growth material. Capillary tubes 34 are preferably filled with hydrophilic growth medium and provide water to growth medium 33 that preferably covers the top surface of floating island 1. Growth medium 33 may be applied to buoyant structure 1 by spraying and curing in place. Perimeter lip 35 helps prevent loss of growth medium 33 due to wave and wind action. Optional matrix scrap pieces 36 may be manufactured into the body of floating island 1 to provide additional surface area for microbial colonization.

In another embodiment shown in FIG. 13, the invention is a floating island that comprises a single bottom layer 41 that is comprised of nonwoven matrix, a middle portion 42 that is comprised of scrap nonwoven matrix or another polymer material and a top blanket 43 of sod, sod-impregnated jute or sod-impregnated polymer blanket. The volume and relative buoyancy of said nonwoven matrix or other polymer material, which may be made of polyester and a polymer other than polyester, determines the volume, if any, of polyurethane foam 44 needed to provide initial buoyancy.

The single layer of nonwoven matrix that comprises bottom layer 41 of this embodiment may be coir, jute, or any polymer, of any thickness. A thinner blanket material is preferred because it is less costly. Middle portion 42 of the floating island, which is made up of scrap matrix or polymer, may have any thickness. Since scrap is less expensive than other materials, this portion of the island is likely to be the thickest portion. Top blanket 43 of the floating island preferably overhangs middle portion 42 and ties into bottom layer 41, providing a sandwich effect that contains the scrap material making up middle portion 42 of the floating island. Polyurethane foam 44 can provide additional buoyancy if needed, as well as an additional means by which to bond all three layers 41, 42 and 43 together.

By cutting scrap polymer into long, thin, jagged strips, and then compressing these strips, the surface area available for microbial colonization can be optimized. These tangled strips are another inexpensive form of matrix blanket. By manipulating the degree of compression of these strips, one may concurrently optimize for plant root growth and gas passage through the strips. In preferred embodiments, the density of these strips is controlled during production by adding a specific volume of strips per square foot, and providing a specific pressure on a compression table. The middle portion of this embodiment may actually be made of another form of matrix blanket. A background art matrix blanket manufactured by Americo requires coating with latex or polyurea or other type coatings to achieve its integrity, whereas this preferred embodiment does not require such coating, but instead relies upon the long narrow strips and jagged edges to provide integrity.

Many variations of the invention will occur to those skilled in the art. Some variations include providing different cross-section thicknesses at different areas within the structure. Other variations call for providing connecting horizontally- and/or vertically-disposed sections within the structure. All such variations are intended to be within the scope and spirit of the invention.

Although some embodiments are shown to include certain features, the applicants specifically contemplate that any feature disclosed herein may be used together or in combination with any other feature on any embodiment of the invention. It is also contemplated that any feature may be specifically excluded from any embodiment of the invention.

1. A structure comprising:
a body that has a center and a perimeter and that comprises a positively buoyant, water-porous and water-permeable matrix material that comprises polymer fibers or polymer shreds that are intertwined to form a randomly oriented blanket having an interior and an exterior, at least a portion of said polymer fibers or polymer shreds being coated with a water-based latex binder, polyurea or polyurethane, said body having a thickened section at its perimeter.

2. The structure of claim 1 wherein said body has a thickened section adjacent to said center.

3. The structure of claim 1 wherein said randomly oriented blanket comprises microbes when in use.

4. A structure for use in a water body having a water surface comprising:
a platform that has a shape, a center and a perimeter and that comprises a positively buoyant, water-porous and water-permeable matrix material, said platform having a thickened section at said perimeter;
wherein, in use, said platform contains a first portion of water that flows through it and a second portion of water that is trapped within said thickened section when said thickened section is lifted above said water surface.

5. (canceled)

6. (canceled)

7. (canceled)

8. A structure comprising:
a first portion that has a perimeter and that comprises a positively buoyant, water-porous and water-permeable matrix material that comprises polyester fibers or polymer shreds that are intertwined to form a randomly oriented blanket, said polyester fibers or polymer shreds being coated with a water-based latex binder, polyurea or polyethylene, said first portion having a thickened section at said perimeter and a center section; and
a second portion that is attached to said center section of said first portion, said second portion being negatively buoyant.

9. (canceled)

10. (canceled)

11. (canceled)

12. A buoyant structure comprising:
a body that comprises a positively buoyant, water-porous and water-permeable matrix material that comprises polymer fibers or polymer shreds that are intertwined to form a randomly oriented blanket, said polymer fibers or polymer shreds being coated with a water-based latex binder, polyurea or polyurethane, said body having an overhanging upper lip section, an undercut center section and an overhanging lower lip section.

13. (canceled)

14. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization.

15. (canceled)

16. (canceled)

17. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization;
wherein said combination is positively buoyant.

18. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization;
wherein said combination is negatively buoyant.

19. (canceled)

20. (canceled)

21. (canceled)

22. (canceled)

23. (canceled)

24. (canceled)

25. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization;
wherein said combination is positively buoyant;
wherein the structure is further comprised of polymer scrap pieces and said polymer scrap pieces are comprised of a combination of polymer fibers and a polymer foam; and
wherein said polymer scrap pieces are comprised of materials having a specific gravity less than 1.0.

26. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization;
wherein said combination is positively buoyant;
wherein the structure is further comprised of polymer scrap pieces and said polymer scrap pieces are comprised of a combination of polymer fibers and a polymer foam; and
wherein said polymer scrap pieces form a combined mixture and polymer scrap pieces are comprised of materials having a range of specific gravities, such that said combined mixture of polymer scrap pieces has a net positive buoyancy.

27. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization;
wherein said combination is positively buoyant;
wherein the structure is further comprised of polymer scrap pieces and said polymer scrap pieces are comprised of a combination of polymer fibers and a polymer foam; and
wherein said polymer scrap pieces are comprised of materials having a specific gravity greater than 1.0.

28. A structure for installation in a body of water having a water surface, said structure comprising:
a plurality of pieces of scrap nonwoven matrix bonded together with a bonding agent or encased in a nonwoven matrix blanket to produce a combination having elements that are operative to provide surfaces for microbial colonization;
wherein said combination is positively buoyant;
wherein the structure is further comprised of polymer scrap pieces and said polymer scrap pieces are comprised of a combination of polymer fibers and a polymer foam; and
wherein said polymer scrap pieces form a combined mixture and said polymer pieces are comprised of materials having a range of specific gravities, such that said combined mixture of polymer scrap pieces has a net negative buoyancy.

29. A structure for installation in an aqueous environment comprising:
a porous containment bag; and
a plurality of pieces of scrap polymer that are encased within said porous containment bag.

30. (canceled)

31. (canceled)

32. A negatively buoyant structure comprising:
a plurality of polymer pieces having a total surface area and a bulk volume and having a total surface area to bulk volume ratio of at least 200 that, together, are operative to provide biomimetic replication of a natural coral formation in saltwater or a stone formation in freshwater, having cavities and crevices for use by aquatic animal life for hiding, resting or feeding.

33. A permeable and negatively buoyant structure for installation in a water body having a bottom, said structure comprising:
a plurality of polymer pieces having a total surface area and a bulk volume and having a total surface area to a bulk volume ratio of at least 200 that, together, are operative to anchor a floating island or to tether another floating object to the bottom, thereby allowing the anchoring of a floating object when the bottom of the water body is soft or otherwise unsuitable for conventional anchors, the permeability of said structure providing additional drag when said object is pulled through the water body, thereby enhancing the anchoring properties of said structure.

34. A floating island for installation in a water body, said floating island comprising:
three first layers, each first layer comprising a nonwoven polymer matrix; and
two second layers, each second layer comprising a plurality of scrap polymer pieces;
wherein each of said second layers is disposed between two of said first layers.

35. The floating island of claim 34 further comprising:
an inlet pipe that is extendable into the water body;
a water pump that is operative to move water into said inlet pipe;
a solar collector that is operative to supply power to said water pump; and
a discharge line for distributing said water over the uppermost of said first layers.

36. A simulated coral reef comprising:
a plurality of scrap polymer pieces that are bonded together to produce a non-buoyant body; and
an injection system for injecting water and/or air into said non-buoyant body.

37. (canceled)

38. (canceled)

39. A polymer scrap structure comprising:
a plurality of scrap polymer pieces that are bonded together with polyurea or polyethylene to form a body having cavities; and
a gas-impermeable top coat comprised of polyurea or polyethylene.

40. (canceled)

41. (canceled)

42. (canceled)

43. (canceled)

44. (canceled)
